(19) United States
(12) Patent Application Publication
(10) Pub. No.: US 2009/0270502 A1
(43) Pub. Date: Oct. 29, 2009

(54) Method for Lowering Risk of Cardiovascular Diseases

(75) Inventors: Kalyana Sundram, Petaling Jaya (MY); Unnikrishnan Ramachandran Unnithan, Johor Darul Takzim (MY); Siew Pheng Foo, Johor Darul Takzim (MY)

Correspondence Address: BIRCH STEWART KOLASCH & BIRCH, PO BOX 747, FALLS CHURCH, VA, 22040-0747, US

(73) Assignees: Carotino Sdn. Bhd., Johor Darul Takzim (MY); Malaysian Palm Oil Board, Kajang, Selangor Darul Ehsan (MY)

(21) Appl. No.: 11/660,031

(22) PCT Filed: Jan. 11, 2007
(86) PCT No.: PCT/MY2007/000003

§ 371 Date: Jul. 30, 2007

(51) Int. Cl. A61K 31/20

(52) U.S. Cl. 514/558

(57) ABSTRACT

The present invention relates to a method for lowering risk of cardiovascular diseases in human, particularly for lowering low density lipoprotein cholesterol (LDL-C) concentration and oxidative stress level while maintaining sufficient high density lipoprotein cholesterol (HDL-C) concentration in human, and to a dietary formulation and a fat or fat blend suitable for human consumption for resulting in the same. The method is by way of controlling daily dietary fat and antioxidants consumption so that: daily dietary fat accounts for 20-40% of the total daily dietary energy intake; 10-20% wt of the fatty acids in daily dietary fat are saturated fatty acids (SFAs); 20-35% wt of the fatty acids in daily dietary fat are polyunsaturated fatty acids (PUFAs); 45-70% wt of the fatty acids in daily dietary fat are monounsaturated fatty acids (MUFAs); and the ratio of antioxidants to polyunsaturated fatty acids (PUFAs) in daily dietary fat is ranging from about 15-70 ppm antioxidants/% wt PUFAs.

### FIELD OF THE INVENTION

The present invention relates to a method for lowering risk of cardiovascular diseases in human, particularly for lowering low density lipoprotein cholesterol (LDL-C) concentration and oxidative stress level while maintaining sufficient high density lipoprotein cholesterol (HDL-C) concentration in human, and to a dietary formulation and a fat or fat blend suitable for human consumption for resulting in the same.

### BACKGROUND OF THE INVENTION

Cardiovascular diseases start from cholesterol build up in arteries. Low density lipoprotein (LDL) is the main source of cholesterol build up in arteries while high density lipoprotein (HDL) helps keep cholesterol from building up in arteries. Excessive total cholesterol (TC) and a high LDL-C to HDL-C ratio (LDL-C/HDL-C) in human are known to cause a higher risk of cardiovascular diseases.

Oxidation of LDL leads to deposition of cholesterol in arteries. Hence, high level of oxidative stress in human is another factor contributing to a higher risk of cardiovascular diseases.

Increasing the proportion of polyunsaturated fatty acids (PUFAs) to saturated fatty acids (SFAs) in daily dietary fat consumption, although beneficial in lowering LDL-C concentration, causes higher level of oxidative stress and lowers HDL-C concentration. Tribble DL in “Antioxidant Consumption and Risk of Coronary Heart Disease: Emphasis on Vitamin C, Vitamin E and β-Carotene: A Statement for Healthcare Professionals from the Nutrition Committee, American Heart Association.” (Circulation. 1999; 99:591-595.) indicates that greater antioxidants intake is associated with lower oxidative stress level and therefore lower disease risk. Grundy et al. in “Dietary Influences on Serum Lipids and Lipoproteins” [J. Lipid Research 31:1149-1172 (1990)] recommend that monounsaturated fats (MONOs), instead of polyunsaturated fats (POLYs), should replace saturated fats (SATs) as much as possible because MONOs do not lower HDL-C concentration like POLYs do and replacing SATs with MONOs will primarily lower LDL-C concentration. These teachings suggest that a high intake of antioxidants in combination with a high proportion of monounsaturated fatty acids (MUFAs) in daily dietary fat consumption is effective in lowering risk of cardiovascular diseases.

U.S. Pat. No. 5,578,334 discloses a method for lowering risk of cardiovascular diseases in human aiming at increasing HDL-C concentration and HDL-C to LDL-C ratio (HDL-C/LDL-C). This patent recommends that 20-40% wt of the fatty acids in daily dietary fat should be made up by SFAs, 15-40% wt by PUFAs with the remaining proportion made up by MUFAs for achieving an optimum HDL-C/LDL-C. It is pointed out in this patent that an excessive proportional intake of PUFAs and MUFAs is avoided to assure a sufficient dietary availability of SFAs which is required for sufficient HDL production. This point indicates that a proportion of SFAs lower than 20% wt in daily dietary fat consumption will render optimum HDL-C/LDL-C unachievable.

### SUMMARY OF THE INVENTION

In the first aspect, the present invention relates to a method for lowering LDL-C concentration and oxidative stress level while maintaining sufficient HDL-C concentration in human by controlling daily dietary fat and antioxidants consumption so that: daily dietary fat accounts for 20-40% of the total daily dietary energy intake; 10-20% wt of the fatty acids in daily dietary fat are saturated fatty acids; 20-35% wt of the fatty acids in daily dietary fat are polyunsaturated fatty acids; 45-70% wt of the fatty acids in daily dietary fat are monounsaturated fatty acids; and the ratio of antioxidants to polyunsaturated fatty acids in daily dietary fat is ranging from about 15-70 ppm antioxidants/% wt PUFAs.

A dietary formulation suitable for resulting in the same contains dietary fat which accounts for 20-40% of the total dietary energy in said dietary formulation and wherein 10-20% wt of the fatty acids in said dietary fat are saturated fatty acids, 20-35% wt are polyunsaturated fatty acids and 45-70% wt are monounsaturated fatty acids and wherein the ratio of antioxidants to polyunsaturated fatty acids in said dietary fat is ranging from about 15-70 ppm antioxidants/% wt PUFAs.

A fat or fat blend suitable for resulting in the same is one wherein 10-20% wt of the fatty acids in said fat or fat blend are saturated fatty acids, 20-35% wt are polyunsaturated fatty acids and 45-70% wt are monounsaturated fatty acids and wherein the ratio of antioxidants to polyunsaturated fatty acids in said fat or fat blend is ranging from about 15-70 ppm antioxidants/% wt PUFAs.

In a second related aspect, the present invention relates to a method for lowering LDL-C to HDL-C ratio and oxidative stress level in human by controlling daily dietary fat and antioxidants consumption so that: daily dietary fat accounts for 20-40% of the total daily dietary energy intake; 10-20% wt of the fatty acids in daily dietary fat are saturated fatty acids; 20-35% wt of the fatty acids in daily dietary fat are polyunsaturated fatty acids; 45-70% wt of the fatty acids in daily dietary fat are monounsaturated fatty acids; and the ratio of antioxidants to polyunsaturated fatty acids in daily dietary fat is ranging from about 15-70 ppm antioxidants/% wt PUFAs.

A dietary formulation suitable for resulting in the same contains dietary fat which accounts for 20-40% of the total dietary energy in said dietary formulation and wherein 10-20% wt of the fatty acids in said dietary fat are saturated fatty acids, 20-35% wt are polyunsaturated fatty acids and 45-70% wt are monounsaturated fatty acids and wherein the ratio of antioxidants to polyunsaturated fatty acids in said dietary fat is ranging from about 15-70 ppm antioxidants/% wt PUFAs.

A fat or fat blend suitable for resulting in the same is one wherein 10-20% wt of the fatty acids in said fat or fat blend are saturated fatty acids, 20-35% wt are polyunsaturated fatty acids and 45-70% wt are monounsaturated fatty acids and wherein the ratio of antioxidants to polyunsaturated fatty acids in said fat or fat blend is ranging from about 15-70 ppm antioxidants/% wt PUFAs.

An example of a fat blend according to the first or second aspect of the present invention is one comprising 10-30% wt red palm oil and 70-90% wt high MUFA oil, preferably canola oil.

In a third related aspect, the present invention relates to a method for lowering LDL-C concentration while maintaining sufficient HDL-C concentration in human by controlling daily dietary fat consumption so that: daily dietary fat accounts for 20-40% of the total daily dietary energy intake; 10-20% wt of the fatty acids in daily dietary fat are saturated fatty acids; 20-35% wt of the fatty acids in daily dietary fat are polyunsaturated fatty acids; and 45-70% wt of the fatty acids in daily dietary fat are monounsaturated fatty acids.

A dietary formulation suitable for resulting in the same contains dietary fat which accounts for 20-40% of the total dietary energy in said dietary formulation and wherein 10-20% wt of the fatty acids in said dietary fat are saturated fatty acids, 20-35% wt are polyunsaturated fatty acids and 45-70% wt are monounsaturated fatty acids.

A fat or fat blend suitable for resulting in the same is one wherein 10-20% wt of the fatty acids in said fat or fat blend are saturated fatty acids, 20-35% wt are polyunsaturated fatty acids and 45-70% wt are monounsaturated fatty acids.

In a fourth related aspect, the present invention relates to a method for lowering LDL-C to HDL-C ratio in human by controlling daily dietary fat consumption so that: daily dietary fat accounts for 20-40% of the total daily dietary energy intake; 10-20% wt of the fatty acids in daily dietary fat are saturated fatty acids; 20-35% wt of the fatty acids in daily dietary fat are polyunsaturated fatty acids; and 45-70% wt of the fatty acids in daily dietary fat are monounsaturated fatty acids.

A dietary formulation suitable for resulting in the same contains dietary fat which accounts for 20-40% of the total dietary energy in said dietary formulation and wherein 10-20% wt of the fatty acids in said dietary fat are saturated fatty acids, 20-35% wt are polyunsaturated fatty acids and 45-70% wt are monounsaturated fatty acids.

A fat or fat blend suitable for resulting in the same is one wherein 10-20% wt of the fatty acids in said fat or fat blend are saturated fatty acids, 20-35% wt are polyunsaturated fatty acids and 45-70% wt are monounsaturated fatty acids.

An example of a fat blend according to the third or forth aspect of the present invention is one comprising 10-30% wt palm oil and 70-90% wt high MUFA oil, preferably canola oil.

The ratio of polyunsaturated fatty acids to saturated fatty acids in any of the above-mentioned methods, dietary formulations and fats or fat blends is preferably ranging from 1 to 3.5.

The saturated fatty acids in any of the above-mentioned methods, dietary formulations and fats or fat blends include at least one of any member of the group consisting lauric acid (12:0), myristic acid (14:0) and palmitic acid (16:0); the polyunsaturated fatty acids include linoleic acid (18:2) and the monounsaturated fatty acids include oleic acid (18:1).

The antioxidants in any of the above-mentioned methods, dietary formulations and fats or fat blends include vitamin E and/or carotenes.

### DETAILED DESCRIPTION OF THE INVENTION

In the first aspect, the present invention provides a method for lowering LDL-C concentration and oxidative stress level while maintaining sufficient HDL-C concentration in human and a dietary formulation and a fat or fat blend suitable for human consumption for resulting in the same.

In a second related aspect, the present invention provides a method for lowering LDL-C to HDL-C ratio (or optimizing HDL-C to LDL-C ratio) and oxidative stress level in human and a dietary formulation and a fat or fat blend suitable for human consumption for resulting in the same.

In a third related aspect, the present invention provides a method for lowering LDL-C concentration while maintaining sufficient HDL-C concentration in human and a dietary formulation and a fat or fat blend suitable for human consumption for resulting in the same.

In a fourth related aspect, the present invention provides a method for lowering LDL-C to HDL-C ratio (or optimizing HDL-C to LDL-C ratio) in human and a dietary formulation and a fat or fat blend suitable for human consumption for resulting in the same.

The method in the first and second aspects is by way of controlling daily dietary fat consumption so that:
daily dietary fat accounts for 20-40% of the total daily dietary energy intake;
10-20% wt of the fatty acids in daily dietary fat are SFAs;
20-35% wt of the fatty acids in daily dietary fat are PUFAs;
45-70% wt of the fatty acids in daily dietary fat are MUFAs; and
the ratio of antioxidants to PUFAs in daily dietary fat is ranging from about 15-70 ppm antioxidants/% wt PUFAs.

The dietary formulation in the first and second aspects contains dietary fat which accounts for 20-40% of the total dietary energy in the dietary formulation wherein 10-20% wt of the fatty acids in said dietary fat are SFAs, 20-35% wt are PUFAs and 45-70% wt are MUFAs and wherein the ratio of antioxidants to PUFAs in said dietary fat is ranging from about 15-70 ppm antioxidants/% wt PUFAs.

The fat or fat blend in the first and second aspects is characterized by having 10-20% wt of its fatty acids made up by SFAs, 20-35% wt made up by PUFAs and 45-70% wt made up by MUFAs and wherein the ratio of antioxidants to PUFAs in said fat or fat blend is ranging from about 15-70 ppm antioxidants/% wt PUFAs. An example of the mentioned fat blend is a blend of 10-30% wt red palm oil and 70-90% wt of a high MUFA oil having at least 50% wt of its fatty acids made up by MUFAs, which in this case is canola oil. In this example, red palm oil is the primary contributor of the antioxidants content, particularly the carotenes content, in the fat blend.

The method in the third and fourth aspects is by way of controlling daily dietary fat consumption so that:
daily dietary fat accounts for 20-40% of the total daily dietary energy intake;
10-20% wt of the fatty acids in daily dietary fat are SFAs;
20-35% wt of the fatty acids in daily dietary fat are PUFAs; and
45-70% wt of the fatty acids in daily dietary fat are MUFAs.

The dietary formulation in the third and fourth aspects contains dietary fat which accounts for 20-40% of the total dietary energy in the dietary formulation wherein 10-20% wt of the fatty acids in said dietary fat are SFAs, 20-35% wt are PUFAs and 45-70% wt are MUFAs.

The fat or fat blend in the third and fourth aspects is characterized by having 10-20% wt of its fatty acids made up by SFAs, 20-35% wt made up by PUFAs and 45-70% wt made up by MUFAs. An example of the mentioned fat blend is a blend of 10-30% wt palm oil and 70-90% wt of a high MUFA oil having at least 50% wt of its fatty acids made up by MUFAs, which in this case is canola oil.

For all the aspects, the dietary fat and the fat or fat blend are preferably derived from plants, e.g. vegetable oils, and the ratio of PUFAs to SFAs (PUFA/SFA) in the dietary fat and the fat or fat blend is preferably ranging from 1 to 3.5. The SFAs include at least one of any member of the group consisting lauric acid (12:0), myristic acid (14:0) and palmitic acid (16:0). The PUFAs include linoleic acid (18:2) while the MUFAs include oleic acid (18:1).

For the first and second aspects, the antioxidants in the dietary fat and the fat or fat blend are primarily vitamin E (tocopherols and/or tocotrienols) and/or carotenes while other antioxidants, including squalene and coenzyme Q10, can also be present. They can be originating from the oil and/or fat used or from other sources which are added to the oil and/or fat used.

Effectiveness of the method of present invention in lowering risk of cardiovascular diseases in human is shown in a non-limiting sense by a clinical test conducted as follows:

On the basis of a questionnaire and a screening examination, 31 volunteers were selected to test the effectiveness of 3 diets: Diet A, B and C specified below, in lowering risk of cardiovascular diseases in human. A volunteer was unable to comply with the prescribed dietary protocol and was subsequently dropped from this test.

The volunteers were healthy Malaysians; they were not taking any medication affecting lipid metabolism, were not on any weight loss programme, were non-smokers, were not consuming alcohol, have no family history of atherosclerosis or hypertension and were normolipemic [Total cholesterol (TC)<6.2 mmol/L; Triglycerides (TG)<2.0 mmol/L].

Diet A was inspired by the teachings of Grundy et al. and it was achieved by using olive oil, which has naturally high content of MUFAs, in preparation of food for consumption by those who followed this diet.

The fatty acids composition and the antioxidants composition in the daily dietary fat consumed by those who followed this diet are as shown in Table 1.

TABLE 1
Diet A

| | | | | | | | | Antioxidants/ | |
|---|---|---|---|---|---|---|---|---|---|
| | | | | | | Antioxidants | | PUFAs in Daily | |
| Fatty Acids Composition | | | | | | Composition in Daily | | Dietary Fat | |
| in Daily Dietary Fat | | | | | | Dietary Fat | | Antioxidants/ | ppm/ |
| SFA | % wt | MUFA | % wt | PUFA | % wt | Antioxidants | ppm | PUFAs | % wt |
| C12:0 | — | C16:1 | 0.9 | C18:2 | 5.9 | α-Tocopherol (T) | 149 | | |
| C14:0 | — | C18:1 | 78.5 | C18:3 | 0.3 | α-Tocotrienol (T3) | — | | |
| C16:0 | 12.1 | | | | | β-Tocotrienol (T3) | — | | |
| C18:0 | 2.3 | | | | | γ-Tocotrienol (T3) | — | | |
| C20:0 | — | | | | | δ-Tocotrienol (T3) | — | | |
| Total | 14.4 | Total | 79.4 | Total | 6.2 | Total T | 149 | T/PUFAs | 24.0 |
| SFA | | MUFA | | PUFA | | Total T3 | — | T3/PUFAs | — |
| | | | | | | Total Vitamin E | 149 | Total Vitamin E | 24.0 |
| | | | | | | (T + T3) | | (T + T3)/PUFAs | |

SFA:MUFA:PUFA = 1:5.5:0.4
PUFA/SFA = 0.4

Diet B was inspired by the teachings of U.S. Pat. No. 5,578,334 and it was achieved by using a blend of 50% wt red palm oil, 35% wt soybean oil and 15% canola oil in preparation of food for consumption by those who followed this diet. The difference between this diet and that of U.S. Pat. No. 5,578,334 is that this diet has high antioxidants content attributed to the use of red palm oil.

The fatty acids composition and the antioxidants composition in the daily dietary fat consumed by those who followed this diet are as shown in Table 2.

TABLE 2
Diet B

| | | | | | | | | Antioxidants/ | |
|---|---|---|---|---|---|---|---|---|---|
| | | | | | | Antioxidants | | PUFAs in Daily | |
| Fatty Acids Composition | | | | | | Composition in Daily | | Dietary Fat | |
| in Daily Dietary Fat | | | | | | Dietary Fat | | Antioxidants/ | ppm/ |
| SFA | % wt | MUFA | % wt | PUFA | % wt | Antioxidants | ppm | PUFAs | % wt |
| C12:0 | 0.7 | C16:1 | 0.4 | C18:2 | 31.4 | α-Tocopherol (T) | 299 | | |
| C14:0 | 0.7 | C18:1 | 35.7 | C18:3 | 0.3 | α-Tocotrienol (T3) | 107 | | |
| C16:0 | 25.7 | | | | | β-Tocotrienol (T3) | 48 | | |
| C18:0 | 3.7 | | | | | γ-Tocotrienol (T3) | 282 | | |
| C20:0 | 1.5 | | | | | δ-Tocotrienol (T3) | 73 | | |
| Total | 32.3 | Total | 36.1 | Total | 31.7 | Total T | 299 | T/PUFAs | 9.4 |
| SFA | | MUFA | | PUFA | | Total T3 | 510 | T3/PUFAs | 16.1 |
| | | | | | | Total Vitamin E | 809 | Total Vitamin E | 25.5 |
| | | | | | | (T + T3) | | (T + T3)/ | |
| | | | | | | | | PUFAs | |
| | | | | | | Carotenes | 363 | Carotenes/ | 11.5 |
| | | | | | | | | PUFAs | |
| | | | | | | Total | 1172 | Total | 37.0 |
| | | | | | | Antioxidants | | Antioxidants/ | |
| | | | | | | | | PUFAs | |

SFA:MUFA:PUFA = 1:1.1:1
PUFA/SFA = 1

Diet C was in accordance with the method of present invention and it was achieved by using a blend of 20% wt red palm oil and 80% wt canola oil in preparation of food for consumption by those who followed this diet.

The fatty acids composition and the antioxidants composition in the daily dietary fat consumed by those who followed this diet are as shown in Table 3.

TABLE 3
Diet C

| | | | | | | | | Antioxidants/ | |
|---|---|---|---|---|---|---|---|---|---|
| | | | | | | Antioxidants | | PUFAs in Daily | |
| Fatty Acids Composition | | | | | | Composition in Daily | | Dietary Fat | |
| in Daily Dietary Fat | | | | | | Dietary Fat | | Antioxidants/ | ppm/ |
| SFA | % wt | MUFA | % wt | PUFA | % wt | Antioxidants | ppm | PUFAs | % wt |
| C12:0 | 0.1 | C16:1 | 0.1 | C18:2 | 20.2 | α-Tocopherol (T) | 275 | | |
| C14:0 | 0.2 | C18:1 | 56.2 | C18:3 | 7.1 | α-Tocotrienol (T3) | 157 | | |
| C16:0 | 11.8 | C20:1 | 1.1 | | | β-Tocotrienol (T3) | 48 | | |
| C18:0 | 2.6 | | | | | γ-Tocotrienol (T3) | 114 | | |
| C20:0 | 0.6 | | | | | δ-Tocotrienol (T3) | 29 | | |
| Total | 15.3 | Total | 56.3 | Total | 27.3 | Total T | 347 | T/PUFAs | 12.7 |
| SFA | | MUFA | | PUFA | | Total T3 | 275 | T3/PUFAs | 10.1 |
| | | | | | | Total Vitamin E | 622 | Total Vitamin | 22.8 |
| | | | | | | (T + T3) | | E (T + T3)/ | |
| | | | | | | | | PUFAs | |
| | | | | | | Carotenes | 145 | Carotenes/ | 5.3 |
| | | | | | | | | PUFAs | |
| | | | | | | Total | 767 | Total | 28.1 |
| | | | | | | Antioxidants | | Antioxidants/ | |
| | | | | | | | | PUFAs | |

SFA:MUFA:PUFA = 1:3.7:1.8
PUFA/SFA = 1.8

The red palm oil used in Diet B and Diet C is naturally riched in carotenes (at least 500 ppm), vitamin E (at least 800 ppm of tocopherols and/or tocotrienols) and other phytonutrients such as squalene, coenzyme Q10, phytosterols and phospholipids.

The volunteers were divided into 3 groups: Group 1, 2 and 3. The groups were required to follow the dietary plan as shown in Table 4.

TABLE 4

| | Dietary Test Period | | | | |
|---|---|---|---|---|---|
| | 1^{st} | | 2^{nd} | | 3^{rd} |
| | 4 Weeks | 1 Week | 4 Weeks | 1 Week | 4 Weeks |
| | (30 Days) | Interval | (30 Days) | Interval | (30 Days) |
| Group | Prescribed Diet | | | | |
| 1 | A | | B | | C |
| 2 | B | | C | | A |
| 3 | C | | A | | B |

Meals for the volunteers were prepared in accordance with the respective diets by a caterer who received detailed instructions from a research dietician about the menu, portion and procedures for control of daily dietary fat consumption. A uniform menu was utilized for all 3 diets but the cooking oil used for meal preparation differed in terms of fatty acids composition and antioxidants composition depending on the prescribed diet.

A common feature of all 3 diets is that the daily dietary fat consumption of the volunteers was controlled so that daily dietary fat accounted for 20-40% of their total daily dietary energy intake. The cooking oil used for meal preparation provided for more than 70% of their daily dietary fat.

Blood samples of 20 ml each were collected before the volunteers started to follow the dietary plan for baseline determination and on the 29^{th} and 30^{th} day of each dietary test period after overnight fasting. The blood samples were first allowed to clot at room temperature for approximately 2 hours, followed by centrifugal separation of plasma at 3000×g for 20 minutes at 4° C. Aliquots of the plasma obtained were utilized for analysis of plasma lipid profile and oxidative stress level. All samples from a particular volunteer were analyzed in a single batch to reduce intra-assay variation.

Plasma TC and TG were determined by enzymatic analysis. HDL-C concentration was determined based on a 2-step methodology: first precipitating LDL and very low density lipoprotein (VLDL) from plasma with dextran and magnesium sulphate, then assaying the remaining supernatant for HDL-C concentration. LDL-C concentration was calculated using the Friedwald Equation [LDL-C=TC−HDL-C−(TG/2.2)].

As an index of dietary compliance, the fatty acids composition of plasma TG was checked for every volunteer at the end of each dietary test period. The fatty acids composition of the meals consumed by the volunteers was also monitored.

The values determined for each volunteer from blood samples collected on the 29^{th} and 30^{th} day of each dietary test period were averaged for statistical analysis. Results of the statistical analysis are shown in Table 5 and 6.

TABLE 5

| Plasma | | Diet | | |
|---|---|---|---|---|
| Lipid Profile | Baseline | A | B | C |
| TC (mmol/L) | 4.98 ± 0.63 | 4.59 ± 0.66 | 4.56 ± 0.56 | 4.36 ± 0.49 |
| TG (mmol/L) | 0.87 ± 0.39 | 0.87 ± 0.30 | 0.92 ± 0.31 | 0.81 ± 0.32 |
| LDL-C | 3.20 ± 0.57 | 2.83 ± 0.62 | 2.80 ± 0.67 | 2.63 ± 0.50 |
| Concentration | | | | |
| (mmol/L) | | | | |
| HDL-C | 1.39 ± 0.36 | 1.37 ± 0.29 | 1.36 ± 0.35 | 1.37 ± 0.28 |
| Concentration | | | | |
| (mmol/L) | | | | |
| LDL-C/HDL-C | 2.49 ± 0.99 | 2.21 ± 0.79 | 2.37 ± 1.33 | 2.04 ± 0.71 |

TABLE 6

| Indices of | | | | |
|---|---|---|---|---|
| Oxidative | | Diet | | |
| Stress Level | Baseline | A | B | C |
| Conjugated | 5.9 ± 2.1 | 5.2 ± 1.7 | 4.6 ± 2.0 | 3.6 ± 1.5 |
| Dienes | | | | |
| ABTS | 39.5 ± 16.3 | 37.5 ± 15.5 | 40.9 ± 14.4 | 39.5 ± 14.9 |

From the plasma lipid profile as shown above, Diet C has outperformed Diet A and Diet B in lowering total cholesterol (TC), triglycerides (TG), LDL-C concentration and LDL-C to HDL-C ratio (LDL-C/HDL-C) while maintaining comparable HDL-C concentration.

Oxidation of lipoproteins involves peroxidation of their PUFAs and yields large amounts of lipid peroxidation products such as conjugated diene peroxides. Peroxidation starts only when antioxidant molecules present in the lipoprotein particle have been consumed. The method used here for screening of antioxidant activity was ABTS radical cation decolourization assay. The pre-formed radical monocation of 2,2′-azinobis-(3-ethylbenzothiazoline-6-sulfonic acid) diammonium salt (ABTS•⁺) was generated by oxidation of ABTS with potassium persulphate and it was reduced in the presence of antioxidants. Reduction of ABTS•⁺ caused its decolourization and the degree of decolourization was measured in terms of decrease in light absorption by spectrophotometer.

From the indices of oxidative stress level as shown above, plasma antioxidant activity of those who followed Diet C was lower compared to those who followed Diet B yet the plasma conjugated dienes level of those who followed Diet C was lower compared to those who followed Diet B. This indicates that oxidative stress level of those who followed Diet B was higher compared to those who followed Diet C.

The inventors of present invention have found the fine balance between SFAs, MUFAs, PUFAs and antioxidants in daily dietary fat consumption which is effective in improving plasma lipid profile and lowering oxidative stress level thus lowering risk of cardiovascular diseases in human.

1) A method for lowering LDL-C concentration and oxidative stress level while maintaining sufficient HDL-C concentration in human by controlling daily dietary fat and antioxidants consumption so that:
daily dietary fat accounts for 20-40% of the total daily dietary energy intake;
10-20% wt of the fatty acids in daily dietary fat are saturated fatty acids;
20-35% wt of the fatty acids in daily dietary fat are polyunsaturated fatty acids;
45-70% wt of the fatty acids in daily dietary fat are monounsaturated fatty acids; and
the ratio of antioxidants to polyunsaturated fatty acids in daily dietary fat is ranging from about 15-70 ppm antioxidants/% wt PUFAs.

2) A dietary formulation suitable for human consumption for lowering LDL-C concentration and oxidative stress level while maintaining sufficient HDL-C concentration wherein said dietary formulation contains dietary fat which accounts for 20-40% of the total dietary energy in said dietary formulation and wherein 10-20% wt of the fatty acids in said dietary fat are saturated fatty acids, 20-35% wt are polyunsaturated fatty acids and 45-70% wt are monounsaturated fatty acids and wherein the ratio of antioxidants to polyunsaturated fatty acids in said dietary fat is ranging from about 15-70 ppm antioxidants/% wt PUFAs.

3) A fat or fat blend suitable for human consumption for lowering LDL-C concentration and oxidative stress level while maintaining sufficient HDL-C concentration wherein 10-20% wt of the fatty acids in said fat or fat blend are saturated fatty acids, 20-35% wt are polyunsaturated fatty acids and 45-70% wt are monounsaturated fatty acids and wherein the ratio of antioxidants to polyunsaturated fatty acids in said fat or fat blend is ranging from about 15-70 ppm antioxidants/% wt PUFAs.

4) A method for lowering LDL-C to HDL-C ratio and oxidative stress level in human by controlling daily dietary fat and antioxidants consumption so that:
daily dietary fat accounts for 20-40% of the total daily dietary energy intake;
10-20% wt of the fatty acids in daily dietary fat are saturated fatty acids;
20-35% wt of the fatty acids in daily dietary fat are polyunsaturated fatty acids;
45-70% wt of the fatty acids in daily dietary fat are monounsaturated fatty acids; and
the ratio of antioxidants to polyunsaturated fatty acids in daily dietary fat is ranging from about 15-70 ppm antioxidants/% wt PUFAs.

5) A dietary formulation suitable for human consumption for lowering LDL-C to HDL-C ratio and oxidative stress level wherein said dietary formulation contains dietary fat which accounts for 20-40% of the total dietary energy in said dietary formulation and wherein 10-20% wt of the fatty acids in said dietary fat are saturated fatty acids, 20-35% wt are polyunsaturated fatty acids and 45-70% wt are monounsaturated fatty acids and wherein the ratio of antioxidants to polyunsaturated fatty acids in said dietary fat is ranging from about 15-70 ppm antioxidants/% wt PUFAs.

6) A fat or fat blend suitable for human consumption for lowering LDL-C to HDL-C ratio and oxidative stress level wherein 10-20% wt of the fatty acids in said fat or fat blend are saturated fatty acids, 20-35% wt are polyunsaturated fatty acids and 45-70% wt are monounsaturated fatty acids and wherein the ratio of antioxidants to polyunsaturated fatty acids in said fat or fat blend is ranging from about 15-70 ppm anti oxidants/% wt PUFAs.

7) A method for lowering LDL-C concentration while maintaining sufficient HDL-C concentration in human by controlling daily dietary fat consumption so that:
daily dietary fat accounts for 20-40% of the total daily dietary energy intake;
10-20% wt of the fatty acids in daily dietary fat are saturated fatty acids;
20 -35% wt of the fatty acids in daily dietary fat are polyunsaturated fatty acids; and
45-70% wt of the fatty acids in daily dietary fat are monounsaturated fatty acids.

8) A dietary formulation suitable for human consumption for lowering LDL-C concentration while maintaining sufficient HDL-C concentration wherein said dietary formulation contains dietary fat which accounts for 20-40% of the total dietary energy in said dietary formulation and wherein 10-20% wt of the fatty acids in said dietary fat are saturated fatty acids, 20-35% wt are polyunsaturated fatty acids and 45-70% wt are monounsaturated fatty acids.

9) A fat or fat blend suitable for human consumption for lowering LDL-C concentration while maintaining sufficient HDL-C concentration wherein 10-20% wt of the fatty acids in said fat or fat blend are saturated fatty acids, 20-35% wt are polyunsaturated fatty acids and 45-70% wt are monounsaturated fatty acids.

10) A method for lowering LDL-C to HDL-C ratio in human by controlling daily dietary fat consumption so that:
daily dietary fat accounts for 20-40% of the total daily dietary energy intake;
10-20% wt of the fatty acids in daily dietary fat are saturated fatty acids;
20-35% wt of the fatty acids in daily dietary fat are polyunsaturated fatty acids; and
45-70% wt of the fatty acids in daily dietary fat are monounsaturated fatty acids.

11) A dietary formulation suitable for human consumption for lowering LDL-C to HDL-C ratio wherein said dietary formulation contains dietary fat which accounts for 20-40% of the total dietary energy in said dietary formulation and wherein 10-20% wt of the fatty acids in said dietary fat are saturated fatty acids, 20-35% wt are polyunsaturated fatty acids and 45-70% wt are monounsaturated fatty acids.

12) A fat or fat blend suitable for human consumption for lowering LDL-C to HDL-C ratio wherein 10-20% wt of the fatty acids in said fat or fat blend are saturated fatty acids, 20-35% wt are polyunsaturated fatty acids and 45-70% wt are monounsaturated fatty acids.

13) A method according to claim 1 wherein the ratio of polyunsaturated fatty acids to saturated fatty acids is ranging from 1 to 3.5.

14) A dietary formulation according to claim 2, wherein the ratio of polyunsaturated fatty acids to saturated fatty acids is ranging from 1 to 3.5.

15) A fat or fat blend according to claim 3, wherein the ratio of polyunsaturated fatty acids to saturated fatty acids is ranging from 1 to 3.5.

16) A method according to claim 1 wherein the saturated fatty acids include at least one of any member of the group consisting lauric acid (12:0), myristic acid (14:0) and palmitic acid (16:0); the polyunsaturated fatty acids include linoleic acid (18:2) and the monounsaturated fatty acids include oleic acid (18:1).

17) A dietary formulation according to claim 2 wherein the saturated fatty acids include at least one of any member of the group consisting lauric acid (12:0), myristic acid (14:0) and palmitic acid (16:0); the polyunsaturated fatty acids include linoleic acid (18:2) and the monounsaturated fatty acids include oleic acid (18:1).

18) A fat or fat blend according to claim 3 wherein the saturated fatty acids include at least one of any member of the group consisting lauric acid (12:0), myristic acid (14:0) and palmitic acid (16:0); the polyunsaturated fatty acids include linoleic acid (18:2) and the monounsaturated fatty acids include oleic acid (18:1).

19) A method according to claim 1 wherein the antioxidants include vitamin E and/or carotenes.

20) A dietary formulation according to claim 2 wherein the antioxidants include vitamin E and/or carotenes.

21) A fat or fat blend according to claim 3 wherein the antioxidants include vitamin E and/or carotenes.

22) A fat or fat blend according to claim 3 comprising 10-30% wt red palm oil and 70-90% wt of a high MUFA oil having at least 50% wt of its fatty acids made up by MUFAs.

23) A fat or fat blend according to claim 9 comprising 10-30% wt palm oil and 70-90% wt of a high MUFA oil having at least 50% wt of its fatty acids made up by MUFAs.

24) A fat or fat blend according to claim 22 wherein the high MUFA oil is canola oil.
